(19) United States
(12) Patent Application Publication
(10) Pub. No.: US 2012/0129785 A1
(43) Pub. Date: May 24, 2012

(54) COMPANION ANIMAL NUTRITION SYSTEM

(75) Inventors: Leslie Fleuranges, Topeka, KS (US); Geoffrey Tolsdorf, Mission Hills, KS (US)

(73) Assignee: Hill's Pet Nutrition, Inc., Topeka, KS (US)

(21) Appl. No.: 13/384,778

(22) PCT Filed: Jul. 21, 2010
(86) PCT No.: PCT/US10/42681

§ 371 Date: Jan. 19, 2012

(51) Int. Cl. A61K 38/02; A61P 9/00; B65D 79/00; A61P 19/00; A23K 1/18; A61P 17/00; A61P 19/02

(52) U.S. Cl. 514/18.6; 514/1; 426/2; 426/108

(57) ABSTRACT

The invention encompasses a nutrition system for a companion animal that includes a plurality of pet food packets, wherein an amount of pet food corresponding to a single meal for a companion animal may be selected and wherein the amount of pet food is formulated to alleviate or manage a disorder in a companion animal. In various embodiments, the pet food packets include the nutritional requirements for a companion animal (e.g., protein, fiber, fat) as well as one or more ingredients that alleviate or manage a disorder in a companion animal.

### CROSS REFERENCE TO RELATED APPLICATIONS

This application claims priority to U.S. Provisional Patent Application No. 61/227,197, filed on 21 Jul. 2009, and to U.S. Provisional Patent Application No. 61/231,175, filed on 4 Aug. 2009, both of which are incorporated herein by reference.

### FIELD OF THE INVENTION

The invention encompasses a nutrition system for a companion animal that includes a plurality of pet food packets, wherein an amount of pet food corresponding to a single meal for a companion animal may be selected and wherein the amount of one or more ingredients of pet food is formulated to alleviate or manage a disorder in a companion animal. In various embodiments, the pet food packets include the nutritional requirements for a companion animal (e.g., protein, fiber, fat) as well as one or more ingredients that alleviate or manage a disorder in a companion animal.

### BACKGROUND OF THE INVENTION

It is an unfortunate fact that many animals, particularly companion animals (e.g., dogs and cats) have disorders that impede their quality of life. For example, an animal having an excess of adipose tissue may be clinically diagnosed as “fat,” “overweight,” or “obese” and, as such, are more likely to suffer from associated physical disorders such as arthritis, heart disease, respiratory disease, diabetes, bladder cancer, hypothyroidism, joint disease and/or pancreatitis.

In addition, when a dog or cat is diagnosed with a disorder it is difficult to have the owner administer medication at appropriate or with necessary frequency to help to alleviate or cure the disorder.

The inventors have developed a nutrition system that includes a feeding regime that can overcome problems associated with unhealthy eating while at the same time providing a healthy food that alleviate or manage a disorder in a companion animal, which is present in a unit meal.

### SUMMARY OF THE INVENTION

The invention encompasses a nutrition system for a companion animal comprising a plurality of pet food packets, wherein an amount of pet food corresponding to a single meal for a companion animal may be selected and wherein the amount of pet food is formulated to alleviate or manage a disorder in a companion animal.

In another embodiment, the invention encompasses a weight loss system for a companion animal in need of losing weight comprising a plurality of pet food packets, wherein an amount of pet food corresponding to a single meal for a companion animal may be selected and wherein the pet food is formulated to affect weight loss in a companion animal.

In another embodiment, the invention encompasses a method for managing nutritional intake of a companion animal comprising: (i) identifying a companion animal in need of nutritional management; (ii) obtaining a plurality of pet food packets, which includes a balanced nutritional amount of a pet food, wherein an amount of pet food corresponding to a single meal for a companion animal may be selected; (iii) administering the contents of at least one pet food packet to a companion animal, wherein the pet food is formulated to alleviate or manage a disorder in a companion animal.

In another embodiment, the invention encompasses a weight loss nutrition system for a companion animal in need of losing weight comprising a plurality of pet food packets, wherein an amount of pet food corresponding to a single meal for a companion animal may be selected and wherein the amount of pet food is formulated to affect weight loss in a companion animal and wherein the pet food comprises a source of protein, fat, fiber, carbohydrate, and at least one ingredient selected from pyruvate, lipoic acid, tomato pomace, myristic acid, flaxseed, fish oil, beta-carotene, and combinations thereof.

### DETAILED DESCRIPTION OF THE INVENTION

The invention generally encompasses a nutrition system for a companion animal comprising a plurality of pet food packets, wherein an amount of pet food corresponding to a single meal for a companion animal may be selected, wherein the amount of pet food is formulated to alleviate or manage a disorder in a companion animal.

In certain embodiments, the companion animal is a dog.

In certain embodiments, the companion animal is a cat.

In certain embodiments, the disorder is a behavioral disorder, bladder disorder, urinary tract disorder, weight disorder, kidney disorder, skin disorder, heart disorder, digestive disorder, gastrointestinal disorder, stomach disorder, small intestinal disorder, large intestinal disorder, arthritis disorder, mobility disorder, liver disorder, a diabetic disorder, eating disorder, cancer disorder, endocrine disorder, haircoat disorder, growth disorder, aging disorder, skeletal disorder, muscle disorder, blood disorder or a combination thereof.

In certain embodiments, the nutrition system further includes companion animal treats, snacks, supplements, and beverages.

In certain embodiments, the nutrition system includes a three to six week supply of pet food packets.

In certain embodiments, the pet food comprises one or more sources of protein, fat, fiber, carbohydrate.

In another illustrative embodiment, the invention encompasses a weight loss system for a companion animal in need of losing weight comprising a plurality of pet food packets, wherein an amount of pet food corresponding to a single meal for a companion animal may be selected, wherein the pet food is formulated to affect weight loss in a companion animal.

In certain embodiments, the companion animal is a dog.

In certain embodiments, the companion animal is a cat.

In certain embodiments, the nutrition system further includes companion animal treats, snacks, supplements, and beverages.

In certain embodiments, the nutrition system includes a three to six week supply of pet food packets.

In certain embodiments, the pet food comprises one or more sources of protein, fat, fiber, carbohydrate.

In another illustrative embodiment, the invention encompasses a method for managing nutritional intake of a companion animal comprising (i)identifying a companion animal in need of nutritional management; (ii) obtaining a plurality of pet food packets, which includes a balanced nutritional amount of a pet food, wherein an amount of pet food corresponding to a single meal for a companion animal may be selected; (iii) administering the contents of at least one pet food packet to a companion animal, wherein the pet food is formulated to alleviate or manage a disorder in a companion animal.

In certain embodiments, the companion animal is a dog.

In certain embodiments, the companion animal is a cat.

In certain embodiments, the disorder is a behavioral disorder, bladder disorder, urinary tract disorder, weight disorder, kidney disorder, skin disorder, heart disorder, digestive disorder, gastrointestinal disorder, stomach disorder, small intestinal disorder, large intestinal disorder, arthritis disorder, mobility disorder, liver disorder, a diabetic disorder, eating disorder, cancer disorder, endocrine disorder, haircoat disorder, growth disorder, aging disorder, skeletal disorder, muscle disorder, blood disorder or a combination thereof.

In certain embodiments, the nutrition system further includes companion animal treats, snacks, supplements or beverages.

In certain embodiments, the nutrition system includes a three to six week supply of pet food packets.

In certain embodiments, the pet food comprises one or more sources of protein, fat, fiber, carbohydrate.

In another illustrative embodiment, the invention encompasses a weight loss nutrition system for a companion animal in need of losing weight comprising a plurality of pet food packets, wherein an amount of pet food corresponding to a single meal for a companion animal may be selected, wherein the amount of pet food is formulated to affect weight loss in a companion animal, and wherein the pet food comprises a source of protein, fat, fiber, carbohydrate, and carnitine.

In addition to alleviating or managing a disorder in a companion animal in certain illustrative embodiments, the pet food packets are formulated to meet a macronutrient diet for one or more meals in a day. In certain illustrative embodiments, the pet food pack of the invention includes a diet to enhance the animal's enjoyment of the food and to provide an increase (perceived or real) in enjoyment or satisfaction by the care giver/owner of the companion animal.

In various embodiments, the companion animals of the invention are the domestic cat (Felis domesticus) or the domestic dog (Canis domesticus). Other companion animals include, fish, bird and horse.

In various illustrative embodiments, different nutrient profiles (nutritional compositions) are included in the pet food packets and include, for example, varying amounts of one or more of fat, protein, carbohydrate, calorie density, fiber, vitamins or minerals.

In accordance with a typical feeding regime for a companion animal, the animal is fed, for example, one meal in the morning and one meal in the afternoon/evening, with optional treats and/or snacks in between. Accordingly, the pet food pack of the invention in various embodiments includes an amount of pet food corresponding to a single meal for a companion animal that may be selected for feeding as the morning meal and another amount of pet food corresponding to a single meal for a companion animal that may be selected for feeding as the afternoon/evening meal. Additional snacks for in between meals or additions to the main meal (e.g., kibbles) may be included.

In an illustrative embodiment, the pet food packet of the invention includes about 20 wt. % to about 40 wt. % of crude protein on a dry matter basis, about 5 wt. % to about 15 wt. % of crude fat on a dry matter basis, about 10 wt. % to about 30 wt. % of total dietary fiber on a dry matter basis, and about 10 wt. % to about 20 wt. % of crude fiber on a dry matter basis.

In another embodiment, the invention encompasses a feline food composition including about 30 wt. % to about 35 wt. % of crude protein on a dry matter basis, about 7.5 wt. % to about 9 wt. % of crude fat on a dry matter basis, about 15 wt. % to about 25 wt. % of total dietary fiber on a dry matter basis, and about 15 wt. % to about 25 wt. % of crude fiber on a dry matter basis.

In certain embodiments, the pet food packet may include a dry, semi-moist or a moist (wet) product. Wet food includes food that is usually sold in tins and has a moisture content of 70% to 90%. Dry food includes food having a similar composition but with 5% to 15% moisture, often presented as small biscuit-like kibbles. Semi-moist food includes food having a moisture content of from above 15% up to 70%. The amount of moisture in any product may influence the type of packaging that can be used or is required.

The pet food packets of the invention encompass any product that a pet consumes in its diet. Thus, the invention encompasses food products as well as pet food snacks (for example snack bars, cereal bars, snacks, biscuits, drinks, and sweet products). In certain embodiments, the food product may be a cooked product.

In certain embodiments, the food product may incorporate meat or animal-derived material (such as beef, chicken, turkey, lamb, fish, blood plasma, marrowbone, etc or one or more thereof).

In certain embodiments, the food product may be meat-free (preferably including a meat substitute such as soya, maize gluten or a soya product) in order to provide a protein source.

In certain embodiments, the food product may contain additional protein sources such as soya protein concentrate, milk, protein, gluten, etc.

In certain embodiments, the food product may also contain a starch source such as one or more grains (e.g. wheat, corn, rice, oats, barley, etc) or may be starch-free.

In certain embodiments, the food product may incorporate or be a gelatinized starch matrix.

In certain embodiments, the food product may incorporate one or more types of fiber such as sugar beet pulp, chicory pulp, chicory, coconut endosperm fiber, wheat fiber etc.

The content of the product/ingredients contributes towards the macronutrient profile of the food. Thus, food products, which according to the invention provide a higher fat content, will be designed accordingly. Dairy products, such as those incorporating a cream or a cheese sauce, may be suitable. The present invention is particularly relevant for a pet food product as described herein which is sold as a pet food, in particular a pet food for a dog or a cat.

The vessels of the food packet may be of any material, shape or size. Their function is, predominantly, to enable ease of use to the care giver/owner of the companion animal and/or the ease of feeding by the companion animal.

In certain embodiments, the pet food packet is labeled to provide information on the meal being administered to the pet. For example, the pet food packet may be labeled “Morning” “Afternoon” or “Evening” to advise the owner when it is appropriate to feed a companion animal.

In another embodiment, the pet food packet is labeled such that the owner of the companion animal is advised of which food packet to administer their companion animal for a particular time. Such a food pack would thus include all of the necessary pet food products for their companion animal for one meal. The food pack therefore allows the owner to simply make available to the companion animal the food products in the food pack for a single meal. This enables ease of use, particularly for owners who take their companion animals away from home (traveling), and those who have busy lives as well as those owners/care givers who do not necessarily know how much food to give to their companion animal.

The food packet according to the invention provides benefits to the companion animal of increased acceptance/palatability increased in enjoyment in feeding and/or increase in enjoyment or satisfaction by the owner of the animal.

In certain embodiments, the invention encompasses a nutrition system that includes packets of food for an individual meal, wherein the packets contain the nutritional requirements necessary for a companion animal as well as an amount of food or an ingredient sufficient to alleviate or manage a disorder in a companion animal.

In one embodiment, the nutrition system includes packets with ingredients that assist in the management or behavior of a dog or cat. The food packet includes ingredients that are formulated to help fight age-related behavior changes in older dogs and cats. For example, without being limited by theory, it is believed that an older cat or dog's brain is more vulnerable to free radicals that are naturally produced by the body. The damage free radicals inflict on the brain is often the cause of age-related behavioral changes in older animals. In certain embodiments, each packet is specifically formulated with an exclusive blend of antioxidants and other nutrients to help fight the signs of brain aging and age-related behavioral changes and even help improve the learning ability of older animals. The nutrition system may further include a composition to assist in the management or behavior.

In another embodiment, the nutrition system includes packets with ingredients that prevent or ameliorate urinary tract disorders of a dog or cat. The food packet includes ingredients that are formulated to prevent the build-up of crystals and stones in the urinary tract that can cause painful and bloody urination and potential blockage in a dog or cat. One type of crystal that forms in a dog's urinary tract is called struvite. The food packet of the invention is formulated specifically to provide nutritional management of dogs with struvite-related urinary tract disease and to reduce levels of magnesium and phosphorus, and also produces an acid urine pH to aid in the management of struvite crystals and stones. The nutrition system may further include a composition to prevent or ameliorate urinary tract disorders.

In another embodiment, the nutrition system includes packets with ingredients that prevent or ameliorate inflammatory skin conditions and food allergy and intolerance in a dog or cat. The food packet includes ingredients that are formulated with enhanced levels of omega-3 fatty acids and antioxidants, which have been shown to be useful for pets with a variety of inflammatory skin conditions that are often associated with excessive itching, scratching and licking. Without being bound by theory, it is believed that food allergy and intolerance may result in numerous skin and stomach problems that can cause discomfort and pain in a dog or cat. This condition, also known as an adverse reaction to food, is usually an abnormal response to the protein found in many pet foods. The pet food packet comes with novel protein to help manage adverse reactions to food. Reactions can occur as a result of beef, dairy products and wheat. The nutrition system may further include a composition to prevent or ameliorate inflammatory skin conditions and food allergies and intolerances.

In another embodiment, the nutrition system includes packets with ingredients that prevent or ameliorate renal hypertension and kidney disease in a dog or cat. The food packet includes ingredients that are formulated to help reduce nutrient excesses that may be harmful to their system. For example, by reducing sodium, dogs with mild heart disease are less likely to exhibit signs associated with sodium and fluid retention. Increasing omega-3 fatty acids and low sodium may help improve blood flow to the kidneys while helping to minimize high blood pressure. The nutrition system may further include a composition to prevent or ameliorate renal hypertension and/or kidney disease.

In another embodiment, the nutrition system includes packets with ingredients that prevent or ameliorate a gastrointestinal disorder (GI) in a dog or cat. The food packet includes ingredients that are formulated to help manage dogs or cats with GI disorders. There are several types of GI disorders that affect dogs or cats, including colitis, constipation/diarrhea, gastroenteritis and pancreatitis. The nutrition system may further include a composition to prevent or ameliorate renal gastrointestinal disorders.

In another embodiment, the nutrition system includes packets with ingredients that maintain healthy joints and improve mobility in a dog or cat. The food packet includes ingredients that are formulated to contain high levels of EPA (Eicosapentanoic Acid), an omega-3 fatty acid found in high concentrations in fish oil, enhanced levels of glucosamine and chondroitin provide the building blocks of healthy cartilage, and L-carnitine helps maintain optimum body weight. The nutrition system may further include a composition to maintain healthy joints and improve mobility.

In another embodiment, the nutrition system includes packets with ingredients that reduce or ameliorate liver problems in a dog or cat. The food packet includes ingredients that are formulated with high quality protein sources that reduce the liver workload and aid in the regeneration of liver tissue. In certain embodiments, added antioxidant nutrients like Vitamin E, Vitamin C and selenium, help combat oxidative stress. The nutrition system may further include a composition to reduce or ameliorate liver problems.

In another embodiment, the nutrition system includes packets with ingredients that reduce or ameliorate problems associated with cancer, chemotherapy or radiation therapy in a dog or cat. The food packet includes ingredients that are formulated for the management of dogs or cats with cancer who are undergoing radiation or chemotherapy and is designed to increase life span and improve the quality of life for dogs or cats with cancer. The nutrition system may further include a composition to reduce or ameliorate problems associated with cancer, chemotherapy or radiation therapy.

In another embodiment, the nutrition system includes packets with ingredients that reduce or ameliorate painful and potentially serious problem is that usually a result of plaque and tartar buildup at and under the gum line of a dog or cat. The food packet includes ingredients that are formulated specifically for the nutritional management of dogs or cats with dental disease. The special fiber matrix scrubs the exposed tooth surface like an edible toothbrush, reducing bacteria-laden plaque. The nutrition system may further include a composition to reduce or ameliorate conditions associated with plaque and tartar build-up.

In another embodiment, the nutrition system includes packets with ingredients that reduce or ameliorate problems associated with excess weight, diabetes and digestive conditions that can cause problems for your dog. The nutrition system may further include a composition to reduce or ameliorate renal problems associated with excess weight, diabetes and digestive conditions.

Feeding the right food can help your pet live a healthier lifestyle. The pet food packet includes a fiber rich formula, may be useful as a nutritional aid for dogs and cats with fiber responsive diseases such as diabetes mellitus, colitis, diarrhea, constipation, and to help manage overweight dogs, including those with struvite urolithiasis.

### EXAMPLES

### Example 1

### Canine Pet Food Packet for Controlling Diabetes, Weight Control and Digestive Problems

An exemplary non-limiting example of a pet food packet for administration to a dog with diabetes, weight control and digestive problems includes dry (e.g., kibble) or wet food including the following ingredients: Ground Whole Grain Corn, Powdered Cellulose 17.1% (source of fiber), Chicken by-product Meal, Chicken Liver Flavor, Soybean Mill Run, Corn Gluten Meal, Soybean Oil, Dried Beet Pulp, Soybean Meal, Iron Oxide, Potassium Chloride, Calcium Sulfate, L-Lysine, Vitamin E Supplement, Choline Chloride, vitamins (L-Ascorbyl-2-Polyphosphate (source of vitamin C), Vitamin E Supplement, Niacin, Thiamine Mononitrate, Vitamin A Supplement, Calcium Pantothenate, Biotin, Vitamin B12 Supplement, Pyridoxine Hydrochloride, Riboflavin, Folic Acid, Vitamin D3 Supplement), minerals (Ferrous Sulfate, Zinc Oxide, Copper Sulfate, Manganous Oxide, Calcium Iodate, Sodium Selenite), Taurine, L-Carnitine, L-Tryptophan, preserved with Mixed Tocopherols & Citric Acid, Beta-Carotene, Rosemary Extract.

### Example 2

### Canine Hypoallergenic Pet Food Packet

An exemplary non-limiting example of a pet food packet for administration to a dog with allergies includes dry (e.g., kibble) or wet food including the following ingredients: Dried Potato Product, Hydrolyzed Chicken Liver, Potato Starch, Soybean Oil (preserved with BHA, propyl gallate and citric acid), Hydrolyzed Chicken, Powdered Cellulose, Calcium Sulfate, Dicalcium Phosphate, Glyceryl Monostearate, Iodized Salt, Choline Chloride, vitamins (Vitamin E Supplement, L-Ascorbyl-2-Polyphosphate (source of vitamin C), Niacin, Thiamine Mononitrate, Vitamin A Supplement, Calcium Pantothenate, Biotin, Vitamin B12 Supplement, Pyridoxine Hydrochloride, Riboflavin, Vitamin D3 Supplement, Folic Acid), Calcium Carbonate, Potassium Chloride, minerals (Ferrous Sulfate, Zinc Oxide, Copper Sulfate, Manganous Oxide, Calcium Iodate, Sodium Selenite), DL-Methionine, Taurine, preserved with Mixed Tocopherols & Citric Acid, Beta-Carotene, Rosemary Extract.

### Example 3

### Canine Bladder Health Pet Food Packet

An exemplary non-limiting example of a pet food packet for administration to a dog with bladder problems includes dry (e.g., kibble) or wet food including the following ingredients: Brewers Rice, Corn Starch, Pork Fat (preserved with mixed tocopherols and citric acid), Dried Egg Product, Powdered Cellulose, Chicken Liver Flavor, Flaxseed, Potassium Citrate, Soybean Oil (preserved with BHA, propyl gallate and citric acid), Calcium Carbonate, L-Lysine, Iodized Salt, Choline Chloride, vitamins (Vitamin E Supplement, Niacin, Thiamine Mononitrate, Vitamin A Supplement, Calcium Pantothenate, Biotin, Vitamin B12 Supplement, Pyridoxine Hydrochloride, Riboflavin, Vitamin D3 Supplement, Folic Acid), Dried Beet Pulp, L-Threonine, Taurine, Vitamin E Supplement, minerals (Ferrous Sulfate, Zinc Oxide, Manganous Oxide, Copper Sulfate, Calcium Iodate, Sodium Selenite), preserved with Mixed Tocopherols & Citric Acid, L-Tryptophan, L-Carnitine, Beta- Carotene, Rosemary Extract.

### Example 4

### Canine Health Pet Food Packet for Treating or Preventing Gum Disease, Plaque, Tartar, and Promoting Fresh Breath

An exemplary non-limiting example of a pet food packet for administration to a dog with gum disease, plaque, tartar, and/or bad breath includes dry (e.g., kibble) or wet food including the following ingredients Brewers Rice, Ground Whole Grain Corn, Chicken By-Product Meal, Powdered Cellulose, Pork Fat (preserved with mixed tocopherols and citric acid), Soybean Mill Run, Soybean Oil, Chicken Liver Flavor, Calcium Sulfate, Potassium Chloride, L-Lysine, Iodized Salt, Choline Chloride, Vitamin E Supplement, vitamins (L-Ascorbyl-2-Polyphosphate (source of vitamin C), Vitamin E Supplement, Niacin, Thiamine Mononitrate, Vitamin A Supplement, Calcium Pantothenate, Biotin, Vitamin B12 Supplement, Pyridoxine Hydrochloride, Riboflavin, Folic Acid, Vitamin D3 Supplement), Taurine, minerals (Ferrous Sulfate, Zinc Oxide, Copper Sulfate, Manganous Oxide, Calcium Iodate, Sodium Selenite), preserved with Mixed Tocopherols & Citric Acid, Beta-Carotene, Rosemary Extract.

### Example 5

### Canine Health Pet Food Packet for Dogs with Cancer and/or Receiving Chemotherapy or Radiation

An exemplary non-limiting example of a pet food packet for administration to a dog with cancer or receiving chemotherapy or radiation includes dry (e.g., kibble) or wet food including Beef By-Products, Water, Pork Liver, Rice, Menhaden Oil, Chicken, Chicken Liver Flavor, Powdered Cellulose, Dried Beet Pulp, Potassium Citrate, Calcium Carbonate, L-Arginine, Iron Oxide, Dicalcium Phosphate, Taurine, Vitamin E Supplement, Choline Chloride, Zinc Oxide, Ferrous Sulfate, Thiamine Mononitrate, Copper Sulfate, Niacin, Manganous Oxide, Calcium Pantothenate, Vitamin B12 Supplement, Pyridoxine Hydrochloride, Biotin, Vitamin D3 Supplement, Riboflavin, Calcium Iodate, Folic Acid, Sodium Selenite.

### Example 6

### Canine Health Pet Food Packet for Dogs with Liver Problems

An exemplary non-limiting example of a pet food packet for administration to a dog with liver problems includes dry (e.g., kibble) or wet food including Rice, Egg Product, Chicken Fat (preserved with mixed tocopherols and citric acid), Soybean Meal, Soybean Oil, Corn Starch, Chicken Liver Flavor, Corn Gluten Meal, Powered Cellulose, Fish Oil, Dicalcium Phosphate, Potassium Chloride, Calcium Carbonate, Dried Beet Pulp, L-Lysine, L-Arginine, Iodized Salt, Choline Chloride, Vitamin E Supplement, Taurine, Zinc Oxide, L-Carnitine, Ascorbic Acid (source of vitamin C), L-Tryptophan, Niacin, Thiamine Mononitrate, Vitamin A Supplement, Beta-Carotene, Calcium Pantothenate, Manganous Oxide, Biotin, Vitamin B12 Supplement, Pyridoxine Hydrochloride, Riboflavin, Folic Acid, Calcium Iodate, Vitamin D3 Supplement, Sodium Selenite, Menadione Dimethylpyrimidinol Bisulfite (source of vitamin K) Supplement, Riboflavin, Calcium Iodate, Folic Acid, Sodium Selenite.

### Example 7

### Canine Health Pet Food Packet for Dogs with Kidney Problems Including Kidney Disease

An exemplary non-limiting example of a pet food packet for administration to a dog with kidney problems includes dry (e.g., kibble) or wet food including Brewers Rice, Pork Fat (preserved with mixed tocopherols and citric acid), Dried Egg Product, Flaxseed, Corn Gluten Meal, Chicken Liver Flavor, Powdered Cellulose, Calcium Carbonate, Dried Beet Pulp, L-Lysine, Potassium Chloride, Potassium Citrate, vitamins (L-Ascorbyl-2-Polyphosphate (source of vitamin C), Vitamin E Supplement, Niacin, Thiamine Mononitrate, Vitamin A Supplement, Calcium Pantothenate, Biotin, Vitamin B12 Supplement, Pyridoxine Hydrochloride, Riboflavin, Folic Acid, Vitamin D3 Supplement), Choline Chloride, Iodized Salt, Calcium Sulfate, Vitamin E Supplement, L-Threonine, Taurine, minerals (Ferrous Sulfate, Zinc Oxide, Copper Sulfate, Manganous Oxide, Calcium Iodate, Sodium Selenite), L-Tryptophan, Magnesium Oxide, preserved with Mixed Tocopherols & Citric Acid, Beta-Carotene, Rosemary Extract.

### Example 8

### Canine Health Pet Food Packet for Maintaining Healthy Joints and Improving Mobility in Dogs

An exemplary non-limiting example of a pet food packet for administration to a dog to maintain healthy joints and improve mobility includes dry (e.g., kibble) or wet food including Ground Whole Grain Corn, Chicken By-Product Meal, Flaxseed, Soybean Mill Run, Brewers Rice, Soybean Meal, Pork Fat (preserved with mixed tocopherols and citric acid), Chicken Liver Flavor, Powdered Cellulose, Fish Oil, Potassium Chloride, L-Lysine, Calcium Carbonate, Choline Chloride, Iodized Salt, DL-Methionine, Vitamin E Supplement, vitamins (L-Ascorbyl-2-Polyphosphate (source of vitamin C), Vitamin E Supplement, Niacin, Thiamine Mononitrate, Vitamin A Supplement, Calcium Pantothenate, Biotin, Vitamin B12 Supplement, Pyridoxine Hydrochloride, Riboflavin, Folic Acid, Vitamin D3 Supplement), L-Threonine, Taurine, Soy Lecithin, Glucosamine Hydrochloride, minerals (Ferrous Sulfate, Zinc Oxide, Copper Sulfate, Manganous Oxide, Calcium Iodate, Sodium Selenite), L-Tryptophan, L-Carnitine, preserved with Mixed Tocopherols and Citric Acid, Chondroitin Sulfate, Beta-Carotene, Rosemary Extract.

### Example 9

### Canine Health Pet Food Packet for Dogs with Gastrointestinal Problems

An exemplary non-limiting example of a pet food packet for administration to a dog with gastrointestinal problems includes dry (e.g., kibble) or wet food including Egg Product, Turkey, Rice, Ground Whole Grain Corn, Pork Liver, Soy Fiber, Dicalcium Phosphate, Potassium Chloride, Iodized Salt, Calcium Carbonate, Choline Chloride, Vitamin E Supplement, Ferrous Sulfate, Zinc Oxide, Taurine, Thiamine Mononitrate, Copper Sulfate, Manganous Oxide, Niacin, Calcium Pantothenate, Vitamin B12 Supplement, Pyridoxine Hydrochloride, Biotin, Riboflavin, Calcium Iodate, Vitamin D3 Supplement, Sodium Selenite, Folic Acid.

### Example 10

### Canine Health Pet Food Packet for Dogs with Behavioral Disorders

An exemplary non-limiting example of a pet food packet for administration to a dog with behavioral disorders or problems includes dry (e.g., kibble) or wet food including Ground Whole Grain Corn, Chicken By-Product Meal, Pork Fat (preserved with mixed tocopherols and citric acid), Brewers Rice, Soybean Mill Run, Soybean Meal, Flaxseed, Fish Meal, Chicken Liver Flavor, Dried Egg Product, Dried Carrots, Dried Spinach, Dried Grape Pomace, Dried Tomato Pomace, Dried Citrus Pulp, Soybean Oil, Potassium Chloride, Vitamin E Supplement, Oat Fiber, Choline Chloride, vitamins (L-Ascorbyl-2-Polyphosphate (source of Vitamin C), Vitamin E Supplement, Niacin, Thiamine Mononitrate, Vitamin A Supplement, Calcium Pantothenate, Vitamin B12 Supplement, Pyridoxine Hydrochloride, Riboflavin, Vitamin D3 Supplement, Folic Acid, Biotin), L-Lysine, Iodized Salt, Calcium Carbonate, L-Tryptophan, Taurine, minerals (Ferrous Sulfate, Zinc Oxide, Copper Sulfate, Manganous Oxide, Calcium Iodate, Sodium Selenite), L-Camitine, preserved with Mixed Tocopherols & Citric Acid, Alpha-Lipoic Acid, Beta-Carotene, Rosemary Extract.

### Example 11

### Canine Health Pet Food Packet for Dogs with Urinary Tract Disorders

An exemplary non-limiting example of a pet food packet for administration to a dog with urinary tract disorders (urinary tract disease is associated with the build-up of crystals and stones in the urinary tract that can cause painful and bloody urination and potential blockage) includes dry (e.g., kibble) or wet food including Beef By-Products, Chicken, Rice, Ground Whole Grain Corn, Pork Liver, Soybean Mill Run, Soybean Oil, Calcium Sulfate, Potassium Chloride, Iodized Salt, Choline Chloride, Vitamin E Supplement, Taurine, Ascorbic Acid (source of vitamin C), Zinc Oxide, Ferrous Sulfate, Thiamine Mononitrate, Beta Carotene, Niacin, Manganous Oxide, Copper Sulfate, Calcium Pantothenate, Vitamin B12 Supplement, Pyridoxine Hydrochloride, Biotin, Vitamin D3 Supplement, Riboflavin, Calcium Iodate, Folic Acid, Sodium Selenite.

### Example 12

### Canine Health Pet Food Packet for Dogs with Skin Disorders

An exemplary non-limiting example of a pet food packet for administration to a dog with skin disorders or problems includes dry (e.g., kibble) or wet food including Rice, Lamb, Lamb Liver, Rice Flour, Powdered Cellulose, Soybean Oil, Fish Oil, Calcium Carbonate, Potassium Chloride, Sodium Tripolyphosphate, Iron Oxide, Dicalcium Phosphate, Choline Chloride, Vitamin E Supplement, Taurine, Ascorbic Acid (source of Vitamin C), Zinc Oxide, Ferrous Sulfate, Beta Carotene, Thiamine Mononitrate, Manganous Oxide, Niacin, Calcium Pantothenate, Vitamin B12 Supplement, Pyridoxine Hydrochloride, Biotin, Vitamin D3 Supplement, Riboflavin, Calcium Iodate, Folic Acid, Sodium Selenite.

### Example 13

### Canine Health Pet Food Packet for Dogs with Heart Conditions

An exemplary non-limiting example of a pet food packet for administration to a dog with heart conditions includes dry (e.g., kibble) or wet food including Chicken, Ground Whole Grain Corn, Rice, Pork Liver, Chicken Fat (preserved with mixed tocopherols and citric acid), Sucrose, Chicken Liver Flavor, Potassium Citrate, Calcium Carbonate, Potassium Chloride, Choline Chloride, Vitamin E Supplement, Taurine, Magnesium Oxide, L-Tryptophan, Ascorbic Acid (source of vitamin C), L-Carnitine, Zinc Oxide, Thiamine Mononitrate, Ferrous Sulfate, Beta-Carotene, Niacin, Calcium Pantothenate, Vitamin B12 Supplement, Manganous Oxide, Copper Sulfate, Pyridoxine Hydrochloride, Biotin, Vitamin D3 Supplement, Riboflavin, Calcium Iodate, Folic Acid, Sodium Selenite.

### Example 14

### Feline Health Pet Food Packet for Cats with Urinary Tract Conditions

An exemplary non-limiting example of a pet food packet for administration to a cat with urinary tract conditions (e.g., Feline Lower Urinary Tract Disease caused by struvite uroliths or urethral plugs, calcium oxalate uroliths, or feline idiopathic cystitis) includes dry (e.g., kibble) or wet food including Brewers Rice, Corn Gluten Meal, Chicken By-Product Meal, Pork Fat (preserved with mixed tocopherols and citric acid), Chicken Liver Flavor, Fish Oil, Potassium Chloride, Calcium Sulfate, Choline Chloride, Vitamin E Supplement, Iodized Salt, Potassium Citrate, vitamins (Vitamin E Supplement, Niacin, Thiamine Mononitrate, Pyridoxine Hydrochloride, Calcium Pantothenate, Riboflavin, Vitamin A Supplement, Biotin, Vitamin B12 Supplement, Folic Acid, Vitamin D3 Supplement), Taurine, minerals (Ferrous Sulfate, Zinc Oxide, Copper Sulfate, Manganous Oxide, Calcium Iodate, Sodium Selenite), preserved with Mixed Tocopherols and Citric Acid, Beta-Carotene, Rosemary Extract.

### Example 15

### Feline Health Pet Food Packet for Cats with Skin Conditions

An exemplary non-limiting example of a pet food packet for administration to a cat with skin conditions includes dry (e.g., kibble) or wet food including Pork Fat (preserved with mixed tocopherols and citric acid), Duck Meal, Soybean Oil (preserved with BHA, propyl gallate and citric acid), Calcium Sulfate, Fish Oil, Natural Flavor, Choline Chloride, DL -Methionine, Iodized Salt, L-Cysteine, Taurine, Dicalcium Phosphate, Vitamin E Supplement, vitamins (L-Ascorbyl-2-Polyphosphate (source of vitamin C), Vitamin E Supplement, Niacin, Thiamine Mononitrate, Vitamin A Supplement, Calcium Pantothenate, Riboflavin, Biotin, Vitamin B12 Supplement, Pyridoxine Hydrochloride, Folic Acid, Vitamin D3 Supplement), Potassium Sulfate, minerals (Ferrous Sulfate, Zinc Oxide, Copper Sulfate, Manganous Oxide, Calcium Iodate, Sodium Selenite), preserved with Mixed Tocopherols & Citric Acid, Beta-Carotene, Rosemary Extract.

### Example 16

### Feline Health Pet Food Packet for Cats with Kidney Problems and Heart Conditions

An exemplary non-limiting example of a pet food packet for administration to a cat with kidney problems and heart conditions includes dry (e.g., kibble) or wet food including Brewers Rice, Corn Gluten Meal, Pork Fat (preserved with mixed tocopherols and citric acid), Chicken By-Product Meal, Soybean Mill Run, Pork Protein Isolate, Chicken Liver Flavor, Calcium Sulfate, Fish Meal, Choline Chloride, Potassium Chloride, Potassium Citrate, Natural Flavor, Iodized Salt, DL-Methionine, Taurine, vitamins (Vitamin E Supplement, Niacin, Thiamine Mononitrate, Vitamin A Supplement, Calcium Pantothenate, Riboflavin, Biotin, Vitamin B12 Supplement, Pyridoxine Hydrochloride, Folic Acid, Vitamin D3 Supplement), minerals (Ferrous Sulfate, Zinc Oxide, Copper Sulfate, Manganous Oxide, Calcium Iodate, Sodium Selenite), preserved with Mixed Tocopherols and Citric Acid, Rosemary Extract.

### Example 17

### Feline Health Pet Food Packet for Cats with Gastrointestinal Conditions

An exemplary non-limiting example of a pet food packet for administration to a cat with gastrointestinal conditions includes dry (e.g., kibble) or wet food including Chicken By-Product Meal, Brewers Rice, Corn Gluten Meal, Ground Whole Grain Corn, Pork Fat (preserved with mixed tocopherols and citric acid), Powdered Cellulose, Dried Chicken, Chicken Liver Flavor, Potassium Chloride, Choline Chloride, Calcium Sulfate, Dried Beet Pulp, DL-Methionine, Iodized Salt, Dried Yeast, minerals (Ferrous Sulfate, Zinc Oxide, Copper Sulfate, Manganous Oxide, Calcium Iodate, Sodium Selenite), Taurine, vitamins (Vitamin E Supplement, Niacin, Thiamine Mononitrate, Vitamin A Supplement, Calcium Pantothenate, Riboflavin, Biotin, Vitamin B12 Supplement, Pyridoxine Hydrochloride, Folic Acid, Vitamin D3 Supplement), preserved with Mixed Tocopherols and Citric Acid, Rosemary Extract.

### Example 18

### Feline Health Pet Food Packet for Cats with Liver Problems

An exemplary non-limiting example of a pet food packet for administration to a cat with liver problems includes dry (e.g., kibble) or wet food including Brewers Rice, Chicken By-Product Meal, Pork Fat (preserved with mixed tocopherols and citric acid), Soybean Meal, Pasta Product, Pork Protein Isolate, Soy Fiber, Dried Egg Product, Fish Meal, Chicken Liver Flavor, Choline Chloride, Soybean Oil, Potassium Chloride, Powdered Cellulose, Taurine, vitamins (Vitamin E Supplement, Thiamine Mononitrate, L-Ascorbyl-2-Polyphosphate (source of vitamin C), Niacin, Vitamin A Supplement, Riboflavin, Calcium Pantothenate, Vitamin B12 Supplement, Biotin, Pyridoxine Hydrochloride, Folic Acid, Vitamin D3 Supplement, Menadione Dimethylypyrimidinol Bisulfite (source of vitamin K)), DL-Methionine, Calcium Sulfate, L-Carnitine, minerals (Zinc Oxide, Manganous Oxide, Ferrous Sulfate, Calcium Iodate, Sodium Selenite), preserved with Mixed Tocopherols and Citric Acid, L-Arginine, Rosemary Extract.

### Example 19

### Feline Health Pet Food Packet for Cats with Diabetes and Associated Disorders

An exemplary non-limiting example of a pet food packet for administration to a cat with diabetes and disorders associated with diabetes includes dry (e.g., kibble) or wet food including Chicken By-Product Meal, Corn Gluten Meal, Pork Fat (preserved with mixed tocopherols and citric acid), Pork Protein Isolate, Powdered Cellulose, Brewers Rice, Ground Whole Grain Corn, Dried Egg Product, Chicken Liver Flavor, L-Lysine, Potassium Chloride, Calcium Sulfate, Choline Chloride, vitamins (L-Ascorbyl-2-Polyphosphate (source of vitamin C), Vitamin E Supplement, Niacin, Thiamine Mononitrate, Vitamin A Supplement, Calcium Pantothenate, Riboflavin, Biotin, Vitamin B12 Supplement, Pyridoxine Hydrochloride, Folic Acid, Vitamin D3 Supplement), Vitamin E Supplement, Taurine, Iodized Salt, minerals (Ferrous Sulfate, Zinc Oxide, Copper Sulfate, Manganous Oxide, Calcium Iodate, Sodium Selenite), L-Carnitine, preserved with Mixed Tocopherols and Citric Acid, Beta Carotene, Rosemary Extract.

### Example 20

### Feline Health Pet Food Packet for Cats with a Weight Disorder

An exemplary non-limiting example of a pet food packet for administration to a cat with a weight disorder includes dry (e.g., kibble) or wet food including Chicken By-Product Meal, Brewers Rice, Corn Gluten Meal, Powdered Cellulose 18.5% (source of fiber), Chicken, Chicken Liver Flavor, Soybean Oil, Calcium Sulfate, Potassium Chloride, Choline Chloride, DL-Methionine, Vitamin E Supplement, Taurine, vitamins (L-Ascorbyl-2-Polyphosphate (source of vitamin C), Vitamin E Supplement, Niacin, Thiamine Mononitrate, Vitamin A Supplement, Calcium Pantothenate, Riboflavin, Biotin, Vitamin B12 Supplement, Pyridoxine Hydrochloride, Folic Acid, Vitamin D3 Supplement), Iodized Salt, minerals (Ferrous Sulfate, Zinc Oxide, Copper Sulfate, Manganous Oxide, Calcium Iodate, Sodium Selenite), L-Carnitine, preserved with Mixed Tocopherols and Citric Acid, Beta-Carotene, Rosemary Extract.

### Example 21

### Feline Health Pet Food Packet for Cats with a Bladder Disorder

An exemplary non-limiting example of a pet food packet for administration to a cat with a bladder disorder includes dry (e.g., kibble) or wet food including Brewers Rice, Chicken By-Product Meal, Pork Fat (preserved with mixed tocopherols and citric acid), Corn Gluten Meal, Dried Egg Product, Calcium Sulfate, Chicken Liver Flavor, Potassium Chloride, Choline Chloride, DL-Methionine, Iodized Salt, Vitamin E Supplement, vitamins (Vitamin E Supplement, Niacin, Thiamine Mononitrate, Pyridoxine Hydrochloride, Calcium Pantothenate, Riboflavin, Vitamin A Supplement, Biotin, Vitamin B12 Supplement, Folic Acid, Vitamin D3 Supplement), Taurine, minerals (Ferrous Sulfate, Zinc Oxide, Copper Sulfate, Manganous Oxide, Calcium Iodate, Sodium Selenite), preserved with Mixed Tocopherols and Citric Acid, Beta-Carotene, Rosemary Extract.

### Example 22

### Feline Health Pet Food Packet for Cats with Plaque, Tartar or a Dental Disorder

An exemplary non-limiting example of a pet food packet for administration to a cat with plaque, tartar or a dental disorder includes dry (e.g., kibble) or wet food including Chicken By-Product Meal, Brewers Rice, Corn Gluten Meal, Ground Whole Grain Corn, Powdered Cellulose 10% (source of fiber), Pork Fat (preserved with mixed tocopherols and citric acid), Chicken Liver Flavor, Calcium Sulfate, Soybean Oil, Potassium Chloride, Choline Chloride, Vitamin E Supplement, DL-Methionine, vitamins (L-Ascorbyl-2-Polyphosphate (source of vitamin C), Vitamin E Supplement, Niacin, Thiamine Mononitrate, Vitamin A Supplement, Calcium Pantothenate, Riboflavin, Biotin, Vitamin B12 Supplement, Pyridoxine Hydrochloride, Folic Acid, Vitamin D3 Supplement), Calcium Carbonate, Iodized Salt, Taurine, minerals (Ferrous Sulfate, Zinc Oxide, Copper Sulfate, Manganous Oxide, Calcium Iodate, Sodium Selenite), preserved with Mixed Tocopherols and Citric Acid, Beta-Carotene, Rosemary Extract.

### Example 23

### Feline Health Pet Food Packet for Cats with Excess Weight, Diabetes and Digestive Problems

An exemplary non-limiting example of a pet food packet for administration to a cat with excess weight, diabetes and digestive problems includes dry (e.g., kibble) or wet food including Chicken By-Product Meal, Corn Gluten Meal, Brewers Rice, Rice Flour, Powdered Cellulose, Chicken, Chicken Liver Flavor, Soybean Oil, Calcium Sulfate, Potassium Chloride, Choline Chloride, Vitamin E Supplement, vitamins (L-Ascorbyl-2-Polyphosphate (source of vitamin C), Vitamin E Supplement, Niacin, Thiamine Mononitrate, Vitamin A Supplement, Calcium Pantothenate, Riboflavin, Biotin, Vitamin B12 Supplement, Pyridoxine Hydrochloride, Folic Acid, Vitamin D3 Supplement), Iodized Salt, Taurine, minerals (Ferrous Sulfate, Zinc Oxide, Copper Sulfate, Manganous Oxide, Calcium Iodate, Sodium Selenite), L-Carnitine, preserved with Mixed Tocopherols and Citric Acid, Beta-Carotene, Rosemary Extract.

### Example 24

### Weight Loss Nutrition System

The example illustrates an illustrative embodiment of the invention that includes an easy to follow system of portion controlled food and treats that helps get a pet to a healthy weight.

Table 1 illustrates an illustrative feeding regimen for small to medium dogs depending on the desired goal weight.

TABLE 1

| | Goal Wt. | Food | Treat | Days Supply | Day Supply |
|---|---|---|---|---|---|
| | (lbs.) | Packets | Packets | of Food | of Treats |
| WLS for Small- | 5 | 1 | 1 | 60 | 72 |
| Med dogs | 10 | 1 | 2 | 60 | 36 |
| 5-40 lbs-Goal | 15 | 1 | 3 | 60 | 24 |
| Weight | 20 | 2 | 1 | 30 | 72 |
| INCLUDES: | 25 | 2 | 3 | 30 | 24 |
| (60) 1.75 oz | 30 | 2 | 4 | 30 | 18 |
| packets of | 35 | 3 | 2 | 20 | 36 |
| specially | 40 | 3 | 4 | 20 | 18 |
| formulated food | | | | | |
| (72) packets of | | | | | |
| great tasting, | | | | | |
| nutritionally | | | | | |
| balance treats- | | | | | |
| each packet | | | | | |
| contains 3 | | | | | |
| medium bones | | | | | |

Table 2 illustrates an illustrative feeding regimen for medium to large dogs depending on the desired goal weight.

TABLE 2

| | Goal Wt. | Food | Treat | Days Supply | Day Supply |
|---|---|---|---|---|---|
| | (lbs.) | Packets | Packets | of Food | of Treats |
| WLS for Med- | 45 | 1 | 3 | 60 | 24 |
| Large dogs | 50 | 2 | 1 | 30 | 72 |
| 45-90 lbs-Goal | 55 | 2 | 1 | 30 | 72 |
| Weight | 60 | 2 | 2 | 30 | 36 |
| INCLUDES: | 70 | 2 | 3 | 30 | 24 |
| (60) 3.50 oz | 80 | 2 | 1 | 20 | 72 |
| packets of | 90 | 3 | 2 | 20 | 36 |
| specially | | | | | |
| formulated food | | | | | |
| (72) packets of | | | | | |
| great tasting, | | | | | |
| nutritionally | | | | | |
| balance treats- | | | | | |
| each packet | | | | | |
| contains 2 large | | | | | |
| bones | | | | | |

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A nutrition system for a companion animal comprising:
a plurality of pet food packets, wherein an amount of pet food corresponding to a single meal for a companion animal may be selected;
wherein the amount of pet food is formulated to alleviate or manage a disorder in a companion animal.

2. The nutrition system of claim 1, wherein the companion animal is a dog.

3. The nutrition system of claim 1, wherein the companion animal is a cat.

4. The nutrition system of claim 1, further comprising a composition to alleviate or manage a disorder in a companion animal.

5. The nutrition system of claim 1, wherein the disorder is a behavioral disorder, bladder disorder, urinary tract disorder, weight disorder, kidney disorder, skin disorder, heart disorder, digestive disorder, gastrointestinal disorder, stomach disorder, small intestinal disorder, large intestinal disorder, arthritis disorder, mobility disorder, liver disorder, a diabetic disorder, eating disorder, cancer disorder, endocrine disorder, haircoat disorder, growth disorder, aging disorder, skeletal disorder, muscle disorder, blood disorder or a combination thereof.

6. The nutrition system of claim 1, wherein the nutrition system further includes companion animal treats, snacks, supplements, and beverages.

7. The nutrition system of claim 1 comprising a three to six week supply pet food packets.

8. The nutrition system of claim 1, wherein the pet food comprises one or more sources of protein, fat, fiber, carbohydrate.

9. A weight loss system for a companion animal in need of losing weight comprising:
a plurality of pet food packets, wherein an amount of pet food corresponding to a single meal for a companion animal may be selected;
wherein the pet food is formulated to affect weight loss in a companion animal.

10. The weight loss system of claim 9, further comprising a composition to affect weight loss in an animal.

11. The weight loss system of claim 9, wherein the companion animal is a dog.

12. The weight loss system of claim 9, wherein the companion animal is a cat.

13. The weight loss system of claim 9, wherein the nutrition system further includes companion animal treats, snacks, supplements or beverages.

14. The weight loss system of claim 9 comprising a three to six week supply pet food packets.

15. The nutrition system of claim 9, wherein the pet food comprises one or more sources of protein, fat, fiber, carbohydrate.

16. A method for managing nutritional intake of a companion animal comprising:
(i) identifying a companion animal in need of nutritional management;
(ii) obtaining a plurality of pet food packets, which include a balanced nutritional amount of a pet food, wherein an amount of pet food corresponding to a single meal for a companion animal may be selected;
(iii) administering the contents of at least one pet food packet to a companion animal;
wherein the pet food is formulated to alleviate or manage a disorder in a companion animal.

17. The nutrition system of claim 16, wherein the companion animal is a dog.

18. The nutrition system of claim 16, wherein the companion animal is a cat.

19. The nutrition system of claim 16, further comprising a composition to affect weight loss in an animal.

20. The nutrition system of claim 16, wherein the disorder is a behavioral disorder, bladder disorder, urinary tract disorder, weight disorder, kidney disorder, skin disorder, heart disorder, digestive disorder, gastrointestinal disorder, stomach disorder, small intestinal disorder, large intestinal disorder, arthritis disorder, mobility disorder, liver disorder, a diabetic disorder, eating disorder, cancer disorder, endocrine disorder, haircoat disorder, growth disorder, aging disorder, skeletal disorder, muscle disorder, blood disorder or a combination thereof.

21. The nutrition system of claim 16, wherein the nutrition system further includes companion animal treats, snacks, supplements or beverages.

22. The nutrition system of claim 16 comprising a three to six week supply pet food packets.

23. A weight loss nutrition system for a companion animal in need of losing weight comprising:
a plurality of pet food packets, wherein an amount of pet food corresponding to a single meal for a companion animal may be selected;
wherein the amount of pet food is formulated to affect weight loss in a companion animal; and
wherein the pet food comprises a source of protein, fat, fiber, carbohydrate, and at least one ingredient selected from camitine, pyruvate, lipoic acid, tomato pomace, myristic acid, flaxseed, fish oil, beta-carotene, and combinations thereof.
